(19) United States
(12) Patent Application Publication
(10) Pub. No.: US 2006/0165602 A1
(43) Pub. Date: Jul. 27, 2006

(54) Method for deterring abuse of opioids by combination with non-release formulation of emetic

(76) Inventors: Bradley S. Galer, West Chester, PA (US); Arnold Gammaitoni, Downingtown, PA (US); Nancy Alvarez, Oxford, PA (US)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 11/361,929

(22) Filed: Feb. 22, 2006

Related U.S. Application Data
(63) Continuation of application No. PCT/US03/25315, filed on Aug. 12, 2003.

(51) Int. Cl. A61K 49/00; A61K 31/485

(52) U.S. Cl. 424/10.1; 514/282

(57) ABSTRACT

The present invention pertains to a pharmaceutical dosage form comprising an opioid and a sequestered emetic. When abuse is attempted by crushing, chewing, or otherwise compromising the sequestration, sufficient emetic is released to cause emesis.

The present invention relates to the use of emetics to deter abuse of drugs, and more particularly relates to deterring the abuse of opioids.

Morphine and other opioids have been known as a very powerful class of analgesic compounds for many years. Their potential as a target of abuse has been known for almost as long. Opioids and their derivatives are used in the pharmaceutical industry as narcotic analgesics, hypnotics, sedatives, anti-diarrheals, anti-spasmotics, and anti-tussives. Opioids are widely used due to their superior, powerful analgesic properties despite well known addictive effects and potential for abuse. As used herein, the term “opioid” includes codeine, dihydrocodeine, hydrocodone, hydromorphone, levorphanol, meperidine, fentanyl, methadone, morphine, oxycodone, oxymorphone, propoxyphene and pharmaceutically acceptable salts, derivatives, and analogs thereof. In the past, abuse of opioids has been generally limited to illicit drugs made in illegal laboratories.

Abuse of pharmaceutical opioids has until recently been relatively limited. Accordingly, action by makers of pharmaceutical opioids would, in the past, have little or no effect on illegal abuse of opioids. The trend has been changing, however, and recently the abuse of pharmaceutical opioids has been increasing. This is especially true in the case of extended release opioid dosage forms. One reason for this is that extended release opioid dosage forms are intended for decreased frequency of dosing, which results in the production of dosage forms having substantially increased amounts of opioid. Therefore, single extended release tablet can provide much more opioid to the potential abuser than past low dose, immediate release dosage forms.

The prior art has addressed the abuse of pharmaceuticals by a variety of techniques. One technique is to incorporate an emetic into the dosage form so that when ingested in sufficient quantity, the attempted abuser vomits the contents of his or her stomach, removing the possibility of the opioid or other drug from being absorbed through the stomach. For example, U.S. Pat. No. 4,175,119 to Porter discloses including an emetic in a pharmaceutical composition to curtail overdosing. The patent describes several emetics, including methyl cephaeline, cephaeline, emetine hydrochloride, psychotrine, O-methylpsychotrine, emetamine, ipecamine, hydro-ipecamine, and ipecacunhic acid. Of these, cephaeline, emetamine, psychotrine, methylpsychotrine, and ipecacunhic acid are all present in ipecac extract. The Porter patent discloses various analgesics useful in the composition, including methadone, meperidine, oxycodone, hydromorphone HCl, codeine, and pentazocine HCl. The emetic chemical is applied as a coating, at a sub-clinical rate and the coated tablet is said to contain 0.25 to 2.0 mg of emetic, where at least about 21 mg (11 to 85 tablets) is needed to induce vomiting. Therefore, if normal prescription directions are followed, no emesis ensues, while ingesting excessive quantities of the coated therapeutic composition will produce emesis.

Other references that include an emetic in an amount sufficient to reduce the potential for abuse or overdose include U.S. Pat. No. 4,269,820 to Davies et al., and U.S. Pat. No. 4,432,787 to Milionis et al. The '820 patent discloses the inclusion of an emetic with a toxic chemical that is not normally intended for oral ingestion. However, if the composition is taken orally, the amount of emetic present in the composition is sufficient to induce emesis in order to protect the individual from potentially toxic substances. The '787 patent discloses a concentrated emetic herbicidal composition, and a method for the preparation thereof. The reference describes the use of an emetic combined with an herbicidal composition in order to protect the individual from toxic material by emesis. Thus if the herbicide is accidentally ingested, it will be disgorged before significant harm occurs. These compositions appear intended to prevent accidental ingestion of potentially dangerous pharmaceuticals. None appear intended to prevent intentional abuse of a single tablet. These prior tablets may also be effective to prevent abuse of pharmaceuticals by intentional ingestion of multiple tablets. However, recent abuse has been predicated on the immediate release of active ingredient from a single extended release tablet. The extended release tablet includes multiple doses (on an immediate release basis) of active ingredient in a single tablet. None of the prior formulations address this problem.

Other formulations are directed to the prevention of intentional abuse of opioid tablets. These formulations take the approach of incorporating an opioid antagonist into the dosage form such that when abuse is attempted, the euphoric “high” of an overdose is blocked. For example, PCT publication No. WO 01/58451 to Oshlack, et al. shows the use of a sequestered opioid antagonist such as naltrexone included in a tablet to prevent abuse of the opioid. The antagonist is sequestered in such a way that the ratio of antagonist released from a crushed tablet to antagonist released from an intact tablet is at least 4:1. This appears intended to remove the incentive to abuse the tablet. If no euphoric effect is achieved, there should be no incentive to abuse the tablet.

Another example of a dosage using the agonist/antagonist technique to limit the possibility of opioid abuse is U.S. Pat. No. 6,274,591 to Foss, et al., which is directed to the use of the opioid methylnaltrexone and related compounds. The method comprises the administration of this compound prior to or simultaneously with the administration of an opioid in order to treat the side effects associated with the use of opioids as analgesics.

However, the effectiveness of orally administered opioid antagonists can be questionable. In the case of naloxone, for example, oral bioavailability is very low. Thus, while naloxone can be effective when the tablet is crushed and taken parenterally, as by snorting or injection, it may be much less effective if the tablet is chewed. Chewing extended release tablets to break the extended release matrix and release all of the opioid at once is becoming a common way of abusing high-dose, extended release opioid tablets. Further, opioid antagonists only serve to counteract opioids and prevent the abuser from obtaining a euphoric effect. They do not prevent the abuser from getting the opioid in his or her bloodstream. The duration of effect of the opioid antagonist may also be shorter than that of the opioid, resulting in an opioid effect after the antagonist wears off. Finally, the use of opioid antagonists requires careful dosing since antagonists are themselves drugs, which have the potential for adverse side effects if administered at too high a dose, including a loss of the intended analgesic effects of the opioid analgesic.

Therefore, in view of the increase in oral abuse of extended release opioid compositions, it would be beneficial to develop a tablet that which would make oral abuse more difficult, less desirable, and aversive for opioid abusers. It would also be beneficial to develop a method which would prevent the absorption of opioid by an abuser, rather than attempting to counteracting or blocking the effects of such absorption of the opioid. The present invention provides such a tablet.

The present invention pertains to a pharmaceutical dosage form comprising an opioid agonist and a sequestered, non-release emetic wherein an effective amount of the emetic is released only when the sequestration is compromised, as when the tablet is crushed or chewed. In a preferred embodiment, the emetic is component of ipecac extract. Preferably, the opioid is contained in a sustained release formulation and is selected from the group consisting of oxycodone, oxymorphone, morphine, and hydromorphone HCl. In a preferred form, the present invention provides a pharmaceutical tablet having an opioid as an active pharmaceutical ingredient contained in a first release matrix, and having a second matrix including a non-release emetic incorporated into the tablet.

The present invention also discloses methods of deterring abuse of a pharmaceutical dosage form by providing an active ingredient susceptible to abuse in a first controlled-release pharmaceutical matrix, and providing a sequestered emetic in a second controlled release pharmaceutical matrix, and releasing the emetic when the sequestration or encapsulation is compromised.

In a preferred embodiment the present invention is directed to an emetic as part of a non-release or slow-release formulation in an opioid tablet. The emetic is most preferably non-release or extremely slow-release in a formulation chosen so that if the tablet is taken as directed, the active emetic does not result in clinically meaningful levels of emetic in the stomach, and does not induce emesis or stomach upset. However, if the medication is crushed or ground, the active emetic is released, resulting in emesis, and preferably vomiting of the contents of the stomach. A tablet or other dosage form made in accordance with the present invention thus prevents oral abuse and creates a negative response, i.e. emesis or discomfort upon abuse, when abused orally. While the present invention is directed at the prevention of oral abuse, by causing the abuser to expel the abused opioid prior to absorption by the body, it can also help deter other types of parenteral abuse, such as injection or intranasal snorting. Such parenteral abuse will cause emesis and discomfort, and while not preventing the absorption of the opioid, the tablet of the present invention will provide negative reinforcement to deter subsequent abuse.

In the tablet of the present invention, an emetic is combined with an opioid tablet in a manner such that the emetic is not orally bioavailable in the formulation, but when crushed or ground the emetic is released and results in vomiting. This formulation prevents actual ingestion and attainment of meaningful serum levels when an attempt is made to abuse the opioids, and by inducing vomiting creates a strong negative response that will tend to condition the abuser against further abuse attempts. As used herein, the term “tablet” is intended to refer to tablets, capsules, and other solid oral dosage forms.

The present invention is most useful slow-release or non-release opioid tablets. The emetic would be chosen so that vomiting is promoted whether the crushed product is ingested orally, parenterally or via intranasal snorting.

The tablet of the present invention can be used with a wide range of opioids. Specifically, it is most preferable to use the tablet of the present invention with opioids having a high potential for abuse. Opioid agonists used in the present invention can be any agonist in general use as an analgesic, including but not limited to, morphine, oxycodone, hydrocodone, codeine, dihydrocodeine, hydromorphone, propoxyphene, methadone, and oxymorphone. Specifically, any addictive opioid in an oral tablet form is the target of the present invention. Most particularly, controlled release oxycodone has recently been the target of abuse and would therefore make a good candidate for use in the present invention. However, while controlled release tablets have been a particular problem lately, the tablet of the present invention may be used for immediate release tablets as well as those in a controlled release format.

The emetic used in the present invention can be any of several well-known emetics, including methyl cephaeline, cephaeline, emetine hydrochloride, psychotrine, O-methylpsychotrine, emetamine, ipecamine, hydro-ipecamine, and ipecacunhic acid. Of these, cephaeline, emetamine, psychotrine, methylpsychotrine, and ipecacunhic acid are all present in ipecac extract. Ipecac extract is widely used and available, as are the individual components thereof. Ipecac extract is derived from the dried roots and rhizomes of G plant.

In the tablet of the present invention, the emetic is contained in a separate matrix from the opioid. That separate matrix can be formed in many different ways. One appropriate configuration is a uniform very slow or non-release matrix with the emetic dispersed therein. The slow release matrix is formulated and granulated into very small granules. These granules are then incorporated into the main matrix of the tablet. In this way, the emetic is contained in a separate slow-release matrix which forms part of the entire tablet. Upon ingestion, the principle matrix of the tablet, which contains the opioid, dissolves, releasing the opioid and also releasing the granules containing the emetic in a solid slow or non-release matrix. The granules then pass through the gastrointestinal tract and out of the body, releasing only minimal emetic, or no emetic at all, without inducing any vomiting.

Another possible configuration for the tablet of the present invention is to incorporate the emetic into an immediate release matrix. The matrix is then granulated and coated with a non-release coating, such as an acrylic polymer. The granules are then incorporated into either an immediate release or a controlled release opioid tablet. Upon administration, the tablet releases opioid at the predetermined rate, but the coated granules release no emetic. Rather, the granules pass through the intestines and are then eliminated from the patient without the induction of vomiting. In this way, the coated granules act as an excipient and, under normal circumstances, have no pharmacological effect whatsoever. Any suitable controlled or immediate release matrix can be used for the emetic, provided that the proper non-release coating is used as well.

Alternatively, a reduced release rate granule can be formed using an immediate release matrix with a reduced release rate coating over the formed granules. Although the description of the invention describes a “non-release” matrix on one embodiment, it is possible that some leakage of emetic may occur where “non-release” is specified. This is acceptable as long as the release rate is very low (lower than necessary to cause emesis). Thus, in the definition of non-release as used herein should be included any reduced release matrix which allows the emetic to be released over a 12-hour period under normal conditions of oral administration at a rate insufficient to cause emesis or stomach upset in a normal patient. Of course, none of the “non-release” matrices described herein are intended to fully encapsulate the emetic so as to prevent release when the tablet is crushed. Furthermore, a suitable non-release coating may be formed by using several known coatings together on a granulated matrix containing emetic. For instance, the granules containing emetic can be covered with a coating which allows for release of material only at a pH below 5 (or 3), which is then covered by a coating which allows release of material only at above a pH of 5 (or 7 or even 9). In that way, when the tablet is ingested, the outer coating will prevent release of emetic while the granules reside in the stomach, and the inner coating will prevent release of emetic once the tablet has passed through the stomach into the intestines, where the pH rises sufficiently to dissolve the outer coating. One skilled in the art would be able to formulate a suitable matrix for use in the tablet of the present invention.

Generally, the amount of emetic used in the tablet of the present invention will not vary with the amount of opioid used (i.e., with the tablet strength). Rather a sufficient amount of emetic should be used to cause swift emesis in a normal patient. This should cause expulsion of the opioid, regardless of the amount of opioid contained in the tablet. However, the amount of emetic in a tablet may be increased if there is a chance that, due to the increased strength of the tablet, abusers may divide the tablet into several smaller doses. In such a case, it would be most desirable to ensure that each dose has sufficient emetic to prevent abuse.

Although certain embodiments of the present invention have been set forth in detail, upon review of the foregoing, those of skill in the art will realize that these embodiments are exemplary in nature. Numerous modifications, adaptations and alternative embodiments will become immediately apparent in view of the description of the invention set forth herein. Thus, in order to ascertain the true scope of the present invention, reference should be made to the appended claims.

What is claimed is:

1. An abuse-resistant pharmaceutical dosage form comprising an opioid in a controlled release formulation and a sequestered emetic.

2. In a controlled release pharmaceutical tablet having an opioid as an active pharmaceutical ingredient, the improvement comprising including in said tablet, a sequestered emetic.

3. The dosage form of claim 1 wherein said sequestered emetic is in a non-release form.

4. The dosage form of claim 3, wherein said sequestered emetic is encapsulated.

5. The dosage form of claim 4 wherein said encapsulated emetic is substantially non-release.

6. The dosage form of claim 1 wherein said emetic is selected from the group consisting of methyl cephaeline, cephaeline, emetine hydrochloride, psychotrine, O-methylpsychotrine, emetamine, ipecamine, hydro-ipecamine, and ipecacunhic acid.

7. The dosage form of claim 1 wherein said emetic is selected from the group consisting of cephaeline, emetamine, psychotrine, methylpsychotrine, and ipecacunhic acid.

8. The dosage form of claim 1 wherein said opioid is selected from the group consisting of morphine sulfate, oxycodone, oxymorphone, hydrocodone, and hydromorphone.
